(19) United States
(12) Patent Application Publication
(10) Pub. No.: US 2024/0358816 A1
(43) Pub. Date: Oct. 31, 2024

(54) FMDV VIRUS-LIKE PARTICLE WITH STABILIZING MUTATION

(71) Applicants: Intervet Inc., Rahway, NJ (US); The Pirbright Institute, Pirbright, Woking, Surrey (GB)

(72) Inventors: Erwin Van Den Born, Wageningen (NL); Ian Jones, Oxford Oxfordshire (GB)

(73) Assignees: Intervet Inc., Rahway, NJ (US); The Pirbright Institute, Pirbright, Woking, Surrey (GB)

(21) Appl. No.: 18/684,529

(22) PCT Filed: Jun. 29, 2022
(86) PCT No.: PCT/EP2022/067888

§ 371 Date: Feb. 16, 2024

(30) Foreign Application Priority Data
Aug. 20, 2021 (EP) 21192323.0

(51) Int. Cl. A61K 39/135; A61K 39/00; A61P 37/04; C07K 14/005; C12N 15/86

(52) U.S. Cl. CPC A61K 39/135; A61P 37/04; C07K 14/005; C12N 15/86; A61K 2039/5258; C12N 2710/14044; C12N 2770/32122; C12N 2770/32123; C12N 2770/32134; C12N 2770/32152; C12N 2770/32171

(57) ABSTRACT

The present invention provides a recombinant foot and mouth disease virus (FMDV) capsid precursor protein comprising a modified VP1 protein and optionally further comprising a modified VP4 protein. The invention further relates to an isolated nucleic acid molecule and an expression vector comprising the nucleic acid molecule for recombinant expression of the modified capsid precursor protein. In further aspects, the invention relates to a virus-like particle (VLP) obtained from the modified capsid precursor protein and a vaccine for use in the protection of a subject against an infection with FMDV produced from the VLP.

The present invention relates to the fields of veterinary medicine and virology. The present invention provides a recombinant foot and mouth disease virus (FMDV) capsid precursor protein comprising a modified VP1 protein and optionally further comprising a modified VP4 protein. The invention further relates to an isolated nucleic acid molecule and an expression vector comprising the nucleic acid molecule for recombinant expression of the modified capsid precursor protein. In further aspects, the invention relates to a virus-like particle (VLP) obtained from the modified capsid precursor protein and a vaccine for use in the protection of a subject against an infection with FMDV produced from the VLP.

### BACKGROUND OF THE INVENTION

Foot-and-mouth disease (FMD) is a highly contagious, acute viral disease of cloven-hoofed, domesticated and wild animals. It is classified as a transboundary animal disease by the Food and Agriculture Organisation of the United Nations (FAO). It is also a notifiable disease. Foot-and-mouth disease is endemic in large parts of Africa, South America, The Middle East and Asia and is, globally, the most economically important infectious disease of livestock, affecting cattle, pigs, sheep, goats and other artiodactyl species like buffalo and deer. FMD was once distributed worldwide but has been eradicated in some regions, including North America and Western Europe. In endemic countries, FMD places economic constraints on the international livestock trade and can be easily reintroduced into disease-free areas unless strict precautions are in place. FMD impacts on the whole livestock industry with loss of income for local farmers.

Current vaccines are made of inactivated virus. Before the virus is inactivated, live FMD virus is produced in high containment facilities, limiting FMD vaccine production. The building costs and maintenance costs of such a facility are higher than that of a conventional facility and due to the limitations that containment brings operating costs are higher as well.

Effective vaccination against FMD requires the presence of intact FMDV capsids (also known as 146S particles) rather than the capsid building blocks that have been proven to be insufficiently immunogenic (Doel and Chong, 1982, Archives of Virology). The inactivated FMD viruses are fragile structures that at acidic pH or at elevated temperatures easily fall apart in the capsid building blocks. Hence, a cold chain is required to deliver effective FMD vaccines to livestock keepers. There is consequently a huge undersupply of vaccine globally, especially in Africa. Therefore, a new vaccine technology for commercial FMD vaccines that can overcome many of the draw backs of the current inactivated virus vaccines is needed.

The virus-like particle (VLP) technology is currently considered one of the few technologies with the potential to be a viable alternative to conventional inactivated vaccines. The benefits of the VLP technology as compared to the current technology are for example higher product stability, greater flexibility in production location (low-containment production), and quicker responses to outbreaks of new strains. VLP-based vaccines are designed as marker vaccines which relieves the necessity of implementing production steps to remove non-structural proteins.

FMDV is a virus of the Picornaviridae family. The virion comprises a single stranded positive sense RNA genome, of about 8 kb, which is contained in a non-enveloped capsid. The capsid is about 30 nm in diameter and has icosahedral symmetry. The capsid consists of a highly regular arrangement of 60 copies of each of four structural viral proteins: VP1, VP2, VP3, and VP4. These are organised in protomer subunits with a sedimentation coefficient of 5S containing one each of VP1-4: five of these protomers form a pentamer of 12S, and the complete capsid consists of 12 pentamers. This can be a non-infectious empty capsid of about 70S to 75S, or a virion of about 146S with the viral RNA genome, which can be infectious.

The FMDV genome encodes a single open reading frame (ORF) that produces a precursor polyprotein that is processed into twelve mature viral proteins, FIG. 1 (from: Balinda et al. Virology Journal 2010, 7:199). The P1 polyprotein intermediate is comprised of four capsid structural proteins, VP1-VP4, sited immediately upstream of the 2A protein which causes non-proteolytic separation of the P1 and P2 polyproteins during translation to release P1-2A from P2. The P1-2A polyprotein is subsequently processed by the FMDV 3C protease into 2A, VP0 (also known as 1AB), VP3 (1C), and VP1 (ID). It is believed that the VP0 protein separates into VP4 and VP2 during encapsulation. FMDV virions are formed by self-assembly from the processed virus structural proteins.

VLPs for use in VLP-based vaccines can be produced by recombinantly expressing FMDV precursor proteins in suitable host cells in analogy to the self-assembly of FMDV virions. The baculovirus expression vector platform is currently used as one of the preferred platforms for the production of VLPs. For example, recombinant expression can be performed in the baculovirus expression system using a modified 3C protease that is less toxic to the insect cells (Porta et al (2013) J Virol Methods). VLPs self-assemble from the processed virus structural proteins, VP0, VP3 and VP1, which are released from the structural protein precursor P1-2A by the action of the virus-encoded 3C protease. Intermediate and non-toxic activity of the 3C enzyme in a P1-2A-3C cassette allows expression and processing of the P1-2A precursor into the structural proteins which assemble into empty capsids.

FMDV is a highly variable agent, and currently has seven main serotypes: O, A, C, SAT (South African territories)-1, SAT-2, and SAT-3, and Asia1. Within these serogroups there are many antigenic variants, subtypes, and quasi-species. Informative is Carrillo et al. (2005, J. Gen. Virol., vol. 79, p. 6487) who have aligned the translated genome sequences of over 100 FMDV isolates from all serotypes. As there is little cross-protection between the main serotypes, typically an FMD vaccine will comprise a separate component for each serotype against which it needs to protect, typically as a combination vaccine.

In respect of prevalence, serotypes A and O have an almost worldwide presence, whereas serotype C has not given any outbreak since 2004. The three SAT serotypes occur in several regions of Africa and the Middle East, and serotype Asia1 in Asia and the Middle East.

The seven serotypes also differ in biophysical properties, mainly in their stability. This is relevant as FMDV, next to being highly contagious, is also quite unstable, and is readily inactivated by heat, acidity, etc. Nevertheless, all FMD vaccines need to be shipped and stored under strict cold-chain logistics. This is a special handicap in the (sub-) tropical- and developing regions of the world where FMD is endemic. In this respect the virions of serotype A are relatively more stable than those of other serotypes, and have a workable shelf-life of 6 months or more. However, serotype O vaccines have a more limited biological half-life, typically only a few months. Even worse is the situation for the three SAT serotypes, for which the notoriously low stability only yield vaccines of low protective capacity, even when administered multiple times.

FMD vaccines made by recombinant DNA expression technology have been investigated for many years. For example, by the expression of FMDV subunits or -epitopes in a variety of systems, such as cell-free expression, or cell-based expression in prokaryotic or eukaryotic cells, including plant cells. Another option is the use of VLP (also called empty FMDV capsids) which are safer to produce than whole virus and were found to be effective immunogens (A. C. Mignaqui et al., 2019, Crit. Rev. Biotechnol., vol. 39 (3), p. 306-320). Such empty capsids can be produced efficiently in a recombinant expression system, such as recombinant Baculovirus using insect cells (Cao et al., 2009, Vet. Microbiol., vol. 137, p. 1: B. M. Subramanian et al., 2012, Antiviral Res. vol. 96 (3), p. 288-95: S. A. Bhat et al., 2013, Vet. Sci. Res. J., vol. 95 (3), p. 1217-23).

Wild-type (unmodified) VLPs, however, cannot be efficiently produced because of their intrinsic instability. They were often found to be even less stable than virions: apparently the viral RNA genome provides some stabilising effect to an FMDV capsid structure.

The FMDV capsid rapidly dissociates into pentamers above physiological temperatures and below physiological pH. For vaccine use this is unfavourable, as the 12S pentamers are immunogenic but are not able to efficiently induce strong virus-neutralizing antibody titres like intact capsids. An approach to improve the thermo- and/or the acid stability of an FMDV capsid is to introduce capsid-stabilizing mutations into one or more of the viral structural proteins.

In particular, the thermostability and resistance to low pH of VLPs can be improved by the introduction of covalent links between the capsid proteins, such as cysteine bridges (WO 2002/000251), or by the introduction of other rationally designed mutations (Porta et al. (2013) PLOS

Pathog). Due to this FMDV capsid stabilization that is linked to the VLP technology, it makes the inclusion of SAT strains into FMD vaccines possible, something that is not straightforward with the conventional vaccine technology due to the labile nature of SAT capsids.

Consequently, the development and improvement of safe, stable and effective FMD vaccines is a continued need.

WO 2002/000251 in particular relates to a modified FMDV P1 antigen comprising a stabilizing mutation, i.e. a substitution of a serine(S) in the wild type sequence of an FMDV A10 strain to cysteine (C) in the modified sequence at amino acid position 179 (corresponding to amino acid position 93 of VP2) of wild-type P1. The modification can also be described as VP2-S093C with 093 corresponding to the amino acid position in the VP2 amino acid sequence at which S is mutated to C. However, despite the mutation at position 93 of the VP2 protein, VLPs based on some serotypes, such as the SAT2 strain, that harbor this substitution (VP2-K093C in SAT2 strains), are still not sufficiently (heat) stable. In addition, yield obtained with VLPs based on these strains harboring the mutation is relatively low.

Thus, there is a need in the art for improved FMDV capsid precursor proteins, which assemble into VLPs with improved stability, in particular thermostability, and which can be obtained with high yield. In addition, it is an object of the present invention to provide effective and safe vaccines against foot-and-mouth disease.

### SUMMARY OF THE INVENTION

In the present invention, it has surprisingly been found that an amino acid modification from threonine to asparagine at position 12 of the VP1 protein, i.e. having the modification VP1-T012N in the amino acid sequence of the FMDV capsid precursor protein, results in FMDV virus-like particles (VLPs) that have improved thermostability and that are produced at higher levels than VLPs including the prior art modification at position 93 of the VP2 protein.

In addition, it has surprisingly been found that the VLP stability and VLP production yield can be further improved in case the VLP is produced from a capsid precursor protein harboring the modification VP1-T012N in combination with an additional amino acid modification from aspartate to glycine at position 053 of the VP4 protein, i.e. having the additional modification VP4-D053G.

Thus, in a first aspect the invention provides a recombinant foot and mouth disease virus (FMDV) capsid precursor protein, comprising at least the virus protein VP1, wherein the VP1 amino acid sequence is modified:
(i) by replacement of amino acid 12 of the amino acid sequence as set forth in SEQ NO: 1 or of the amino acid corresponding to amino acid 12 of the amino acid sequence as set forth in SEQ NO: 1 by an asparagine.

In a particularly preferred embodiment, the invention provides a recombinant foot and mouth disease virus (FMDV) capsid precursor protein, comprising the virus protein VP1, wherein the VP1 amino acid sequence is modified:
(i) by replacement of amino acid 12 of the amino acid sequence as set forth in SEQ NO: 1 or of the amino acid corresponding to amino acid 12 of the amino acid sequence as set forth in SEQ NO: 1 by an asparagine, and wherein the capsid precursor protein further comprises the virus protein VP4, wherein the VP4 amino acid sequence is modified:
(ii) by replacement of amino acid 53 of the amino acid sequence as set forth in SEQ NO: 2 or of the amino acid corresponding to amino acid 53 of the amino acid sequence as set forth in SEQ NO: 2 by a glycine.

The modification (i) is at amino acid position 12 of the VP1 region of wild-type FMDV strains, such as SAT2/ETH/65/2009 or SAT2/SAU/6/2000, changing the amino acid threonine to asparagine. Thus, the modification will be designated herein as “VP1-T012N”, with “T” and “N” identifying the amino acid change from threonine to asparagine in the VP1 protein, and the digit “012” identifying the position of the modification in the VP1 amino acid sequence.

The modification (ii) is at amino acid position 053 of the VP4 region of wild type FMDV strain, such as SAT2/ETH/65/2009 or SAT2/SAU/6/2000, changing the amino acid aspartate to glycine. Thus, the modification will also be designated herein as “VP4-D053G”, with “D” and “G” identifying the amino acid change from aspartate to glycine in the VP4 protein, and the digit “053” identifying the position of the modification in the VP4 amino acid sequence.

SEQ ID NO: 1 describes the amino acid sequence of the VP1 protein of the wild-type strain FMDV SAT2/SAU/6/2000. SEQ ID NO:2 describes the amino acid sequence of the VP4 protein of the wild-type strain FMDV SAT2/SAU/6/2000. However, the invention is not limited to a VP1 or VP4 protein or a capsid precursor protein, such as the P1 protein, comprising the VP1 protein, and optionally the VP4 protein, of this particular strain, which is merely used to identify the position of the amino acid modifications in the amino acid sequences of the VP1 and VP4 proteins. Due to natural sequence variation between different FMDV strains, these positions can be different in other FMDV strains, as will be described herein below. Hence, the invention also covers an FMDV capsid precursor protein comprising the VP1 and optionally the VP4 protein of other FMDV strains, even though the position of the modifications might differ from the position according to SEQ ID NO: 1 and SEQ ID NO:2. The corresponding position of the amino acid modifications (i) and (ii) in other FMDV strains can be found, for example, via aligning the VP1 and VP4 amino acid sequences of different FMDV strains.

Thus, the present invention in the first aspect provides a recombinant FMDV capsid precursor protein comprising at least the VP1 protein harboring the amino acid modification (i) VP1-T012N.

In a preferred embodiment of the first aspect, the invention provides a recombinant FMDV capsid precursor protein further comprising the VP4 protein harboring the amino acid modification (ii) VP4-D053G.

In a second aspect, the invention provides an isolated nucleic acid encoding the recombinant FMDV capsid precursor protein of the first aspect.

In a third aspect, the invention provides an expression vector comprising the nucleic acid sequence of the second aspect encoding the recombinant FMDV capsid precursor protein.

In a fourth aspect, the invention provides a method of producing FMDV virus-like particles (VLP) in a recombinant expression system, the method comprising:
(i) infecting a host cell with the expression vector according to the third aspect, wherein the host cell is capable of recombinantly producing the VLP,
(ii) culturing the host cell under conditions under which the host cell produces the FMDV VLP, and
(iii) harvesting FMDV VLP produced by the host cell from the cell culture.

In a fifth aspect, the invention provides a vaccine for use in the protection of a subject against an infection with FMDV, the vaccine being obtainable by a method according to the fourth aspect.

In a sixth aspect, the invention provides a method of protecting a subject against an infection with FMDV, which comprises the step of producing an FMDV VLP by a method according to the fourth aspect, incorporating the VLP into a vaccine by addition of a pharmaceutically acceptable carrier, and administering the vaccine to the subject.

In a seventh aspect, the invention provides a vaccine comprising an FMDV VLP produced from a recombinant protein according to the first aspect.

### DETAILED DESCRIPTION

### Definition of Terms

A “capsid precursor protein” is a structural protein, which takes part in the formation of a virus capsid or of a building block thereof. FMDV capsid precursor proteins typically comprise the structural protein P1. Most preferably, the FMDV capsid precursor protein at least comprises the P1 and 2A proteins (also referred to herein as P1-2A capsid precursor).

A “capsid precursor protein P1” of the invention refers to the FMDV structural protein processed by the FMDV 3C protease (3Cpro) into the mature VP0, VP3, and VP1 proteins. The capsid precursor protein P1 protein may also be referred to as polyprotein or proprotein. In the context of the present invention, the FMDV capsid precursor protein P1 typically comprises at least the proteins VP1, VP2, VP3 and VP4. Alternatively, the FMDV capsid precursor protein may comprise one or more of the proteins VP1, VP2, VP3 and VP4. The FMDV capsid precursor protein may also comprise the protein VP0 comprising the proteins VP2 and VP4.

A “VP0 protein”, “VP1 protein”, “VP2 protein”, “VP 3 protein”, and “VP 4 protein” of the invention refers to the viral protein number 0, 1, 2, 3 or 4 of FMDV, which are known as structural proteins of an FMDV capsid. As the skilled person readily appreciates, the variability that is inherent to FMDV means that variations in size and amino acid sequence of these structural proteins will occur in nature. The amino acid sequences of these structural proteins from a large number of FMDV isolates are publicly available from sequence databases such as GenBank™, or Swiss Prot™.

A “modification” is a replacement of one element for another; for the invention this is a mutation which regards the replacement of one amino acid or nucleic acid by another, depending on whether the subject is a protein, a DNA or an RNA molecule. The element that is replaced is the element that occurs in the unmodified parental, or wildtype version of the protein or nucleic acid. As a result, a modification according to the invention leads to a capsid precursor protein P1 that differs from its parental, or wildtype form.

To serve as a reference for the invention, “SEQ ID NO: 3” presents the amino acid sequence of the capsid precursor protein P1 of FMDV strain SAT2/SAU/6/2000, derived from GenBank accession number: AY297948. The capsid precursor protein P1 is provided at the N-terminus with a methionine (M) to reflect the recombinantly produced version of this protein described in the invention. The following numbering includes the added M as amino acid 1 and specifies the protein sections of VP proteins for FMDV strain SAT2/SAU/6/2000. The VP0 protein is the section of amino acids no. 1-305 of the complete P1 polyprotein, and which is processed into the separate protein VP4 and VP2. The VP1 protein is the section of amino acids no. 528-741 of the complete P1 polyprotein (described herein as SEQ ID NO:1). VP2 protein is the section of amino acids no. 87-305 of the complete P1 polyprotein. The VP3 protein is the section of amino acids no. 306-527 of the complete P1 polyprotein. The VP4 protein is the section of amino acids no. 1-86 of the complete P1 polyprotein (described herein as SEQ ID NO:2).

The inherent variability of FMDV means that the position of the modifications within the capsid precursor protein P1 of other FMDV isolates or serotypes is not in the exact same position, e.g. it can be offset by one or more amino acids, in either the N-terminal or C-terminal direction. Nevertheless, the exact position within the nucleic acid or amino acid sequence can be easily identified using, for example, a standard computer program for molecular-biological analysis such as sequence alignment tools. Consequently, for the invention the amino acid position numbers of the capsid precursor protein P1 are specified relative to SEQ ID NO: 3, but in different FMDV isolates these may be located at different position numbers.

It has surprisingly been observed that the present invention is most beneficial in FMDV strains from the SAT serotype(s). Without being limited thereto, FMDV strains for use in the invention are thus preferably FMDV strains from SAT-1, SAT-2, or SAT-3 serotype(s), as for these serotypes stability issues have the most impact in the field. Most preferably, the FMDV strain is of the SAT-2 serotype.

Preferred FMDV strains are those that are recommended by the World Reference Laboratory for Foot-and-Mouth Disease (WRL-FMD) as high priority vaccine candidates. The recommendations are published by WRL-FMD on a quarterly basis.

A “virus-like particle” (VLP), which may also be referred to in the art as “empty capsid”, is an entity which comprises the protein shell of a virus but lacks the RNA or DNA genome. A VLP should be antigenic and immunogenic in the same way as the wild-type virus because it retains the same structural epitopes, but it should produce no infection, due to the lack of the virus genome.

An FMDV VLP is typically formed from the P1-2A capsid precursor. As described above, the 2A protease cleaves itself at its C terminus to release P1-2A from P2. Processing of the P1-2A capsid precursor is affected by the 3C protease to produce 2A and the capsid proteins VP0, VP3 and VP1. The VLP is formed by self-assembly from these capsid proteins.

VLPs may be produced using the baculovirus expression system using a modified 3C protease that is less toxic to the insect cells (Porta et al., 2013, J Virol Methods). Intermediate and non-toxic activity of the 3C enzyme in a P1-2A-3C expression cassette allows recombinant expression and processing of the P1-2A precursor into the structural proteins, VP0, VP1, and VP3, which assemble into VLPs. The production of VLPs may be investigated or verified using techniques known in the art such as sucrose density centrifugation or electron microscopy. Monoclonal antibodies specific for conformational epitopes on the wild-type virus may be used to investigate whether the structure and antigenicity of the empty capsid is retained.

The term “nucleic acid sequence” includes an RNA or DNA sequence. It may be single or double stranded. It may, for example, be genomic, recombinant, mRNA or cDNA.

The term “isolated” is to be interpreted as: isolated from its natural context, by deliberate action or human intervention: e.g. by an in vitro procedure for biochemical purification.

Typically, a “nucleic acid” or “nucleic acid molecule” encoding a protein, here: a modified FMDV capsid precursor protein P1 according to the invention, is an open reading frame (ORF), indicating that no undesired stop-codons are present that would prematurely terminate the translation into protein. For the invention the nucleic acid molecule typically encodes the complete capsid precursor protein P1. In an alternative embodiment of the invention, the P1 coding sequence may be split up into multiple expression units, which are expressed as separate recombinant proteins for the assembly of VLPs. For example, the capsid precursor proteins required for the assembly of FMDV VLPs may be expressed separately, for example by recombinantly producing VP1, VP2, VP3 and VP4, or recombinantly producing VP0, VP1 and VP3. Thus, in the present invention FMDV VLPs may be obtained either from the recombinant P1 capsid precursor protein, which comprises at least the VP1 protein containing the modification (i) and optionally further comprises the VP4 protein containing the modification (ii) as described herein, or may be obtained by separately expressing the VP1 and/or the VP4 recombinant proteins, and further recombinantly expressing all other VP proteins necessary for the assembly of VLPs as separate entities.

For the present invention, the exact nucleotide sequence of a nucleic acid molecule according to the invention is not critical, provided the nucleotide sequence allows the expression of the desired amino acid sequence, here: the desired FMDV VP1 protein or the desired VP1 and the VP4 proteins, or the capsid precursor protein P1 comprising the VP1 and optionally the VP4 protein. However, as is well known in the art, different nucleic acids can encode the same protein due to the ‘degeneracy of the genetic code’.

For the present invention, a nucleic acid molecule can be a DNA or an RNA molecule. This depends on the source material used for its isolation, and on the intended use. The skilled person is well aware of methods to isolate one or the other type of molecule from a variety of starting materials, and of methods to convert one type into the other.

An “isolated nucleic acid molecule” according to the invention can conveniently be manipulated in the context of a vector, such as a DNA plasmid, when it is in DNA form. To allow an isolated nucleic acid molecule according to the invention to actually express the modified FMDV capsid precursor protein P1 according to the invention, it will require proper expression control signals and a suitable environment. For example, a nucleic acid molecule needs to be operatively linked to an upstream promoter element, and needs to contain a translation start at the beginning of the coding sequence and a translation stop at the end of the coding sequence. In addition, translational enhancers can be included upstream and/or downstream of the coding region to increase expression levels. Typically, the plasmids and vectors used in the context of a particular expression system will provide for such elements and enhancers. Also, the bio-molecular machinery for transcription and translation is typically provided by a host cell used for such expression. By modifying the various elements and enhancers, the expression of the capsid precursor protein P1 according to the invention can be optimised in e.g. timing, level, and quality: all this is within the routine capabilities of the skilled person. Therefore, in a preferred embodiment, the isolated nucleic acid molecule according to the invention in addition comprises expression control signals. A recombinant expression system for use in the invention typically employs a host cell, which can be cultured in vitro. Well known in the art are host cells from bacterial, yeast, fungal, plant, insect, or vertebrate cell expression systems. Alternatively, in vivo expression systems may be used, employing (transgenic) animals, plants or insects for recombinant gene expression (He et al., 2020, Arch Virol 165:2301-2309, doi.org/10.1007/s00705-020-04754-9).

A “translational enhancer” is a nucleotide sequence forming an element, which can promote translation and, thereby, increase protein production. Typically, a translational enhancer may be found in the 5′ and 3′ untranslated regions (UTRs) of mRNAs. In particular, nucleotides in the 5′-UTR immediately upstream of the initiating ATG codon of the gene of interest (GOI) may have a profound effect on the level of translation initiation.

An “expression vector” (syn. “expression construct”), is usually a plasmid or virus designed for recombinant gene expression in cells. The vector is used to introduce a specific gene into a target cell, and can commandeer the cell's mechanism for protein synthesis to produce the protein of interest (POI) encoded by the gene. In order to express the recombinant gene to produce the POI, the expression vector typically comprises at least a promotor to drive the expression of the GOI and may further comprise one or more translational enhancers to increase the yield of the POI.

A “baculovirus expression vector” is an expression vector based on a baculovirus, which is used for recombinant gene expression in a host cell, such as an insect cell. Baculovirus expression systems are established in the art and are commercially available, such as the Bac-to-Bac expression system (ThermoFisher Scientific, Germany). In these baculovirus expression systems, the naturally occurring polyhedrin gene within the wild-type baculovirus genome is typically replaced with a recombinant gene or cDNA. These genes are commonly under the control of the polyhedrin or p10 baculovirus promoters.

The most common baculovirus used for gene expression is Autographa californica nucleopolyhedrovirus (AcNPV). AcNPV has a large (130 kb), circular, double-stranded DNA genome. The GOI is cloned into a transfer vector containing a baculovirus promoter flanked by baculovirus DNA derived from a nonessential locus, such as the polyhedrin gene. The recombinant baculovirus containing the GOI is typically produced by homologous recombination, such as in insect cells, between the transfer vector and the genome of the parent virus (such as AcNPV).

The term “vaccine” as used herein refers to a preparation which, when administered to a subject, induces or stimulates a protective immune response. A vaccine can render an organism immune to a particular disease.

To “protect an animal against an infection with FMDV” means aiding in preventing, ameliorating or curing a pathogenic infection with FMDV, or aiding in preventing, ameliorating or curing a disorder arising from that infection, for example to prevent or reduce one or more clinical signs resulting from a post treatment (i.e. post vaccination) infection with FMDV.

The term “prevention” or “preventing” is intended to refer to averting, delaying, impeding or hindering the FMDV infection by a prophylactic treatment. The vaccine may, for example, prevent or reduce the likelihood of an infectious FMDV entering a host cell.

### DETAILED DESCRIPTION OF THE INVENTION

In a first aspect, the invention provides a recombinant foot and mouth disease virus (FMDV) capsid precursor protein comprising at least the VP1 protein, wherein the amino acid sequence of the VP1 protein as set forth in SEQ ID NO: 1 is modified at position 012 from threonine to asparagine (designated herein as “modification (i) VP1-T012N”).

In a preferred embodiment of the first aspect, the FMDV capsid precursor protein further comprises the VP4 protein, wherein the amino acid sequence of the VP4 protein as set forth in SEQ ID NO: 2 is modified at position 053 from aspartate to glycine (designated herein as “modification (ii) VP4-D053G”).

It has surprisingly been found in the present invention that the modification (i) VP1-T012N, in particular when used in combination with the modification VP4-D053G, provides an improved capsid stability and gives higher expression levels resulting in more VLPs produced compared to a comparative strain harboring the prior art modification at position 93 of the VP2 protein (i.e. VP2-S093C in the A10 strain, or VP2-K093C in SAT2 strains), which changes the amino acid at position 93 of the amino acid sequence in the wild type strain to cysteine, as described in WO 2002/000251.

In a further preferred embodiment, the capsid precursor protein does not contain the modification at position 93 of the wild type VP2 amino acid sequence described in WO 2002/000251, i.e. the capsid precursor protein does not contain a cysteine at position 093 of the VP2 protein.

In particular, it has surprisingly been found in the present invention that no further improvement in thermostability and yield is achieved when the amino acid modifications VP1-T012N and optionally VP4-D053G of the capsid precursor protein are used in combination with the modification VP2-S093C described in WO 2002/000251 (VP2-K093C in SAT2 strains). Further, it has been shown in the present invention that a combination of the amino acid modifications VP1-T012N and VP4-D053G outperforms the modification VP2-K093C in terms of thermostability and yield.

The first modification (i) designated herein as VP1-T012N is characterized in that the amino acid threonine is replaced by asparagine at position 12 of the capsid protein VP1 in the wild-type strain SAT2/ETH/65/2009.

In particular, the modification VP1-T012N is obtained by replacing, in position 12 of amino acids sequence SEQ ID NO: 1 of the VP1 protein, an amino acid of the original sequence with an asparagine. As a general rule, the position of this amino acid is identical in the other FMDV strains (as is the case particularly with the strains described in the examples). In the sequence of other FMDV strains, the position may possibly vary slightly and may be 11 or 13, for example.

To identify or confirm the amino acid which is to be modified, the amino acid sequence of this region of several FMDV strains are aligned with the corresponding region (for example of the order of about ten or slightly more—e.g. 10 to 20-amino acids) of the sequence in SEQ ID NO: 1, taking into account the fact that the sequences are well conserved in structure among the different foot-and-mouth viruses. It was found, in particular, by comparing the sequences of different FMDV strains, that the region may be written as follows:

Gly Glu X₃ Ala X₅ X₆ Val X₈ X₉ X₁₀

where
X₃ is Ser or Gly
X₅ is Asp or Glu
X₆ is Pro or Val
X₈ is Thr
X₉ is Thr, Ala, Val or Serine
X₁₀ is Thr or Asp
   wherein the modification is in X₈, which is modified in the invention to asparagine.

The modification (ii) at position 053 of the wild-type amino acid sequence of SEQ ID NO: 2 is designated herein as VP4-D053G and characterized in that the amino acid aspartate is replaced by glycine at position 53 of the capsid protein VP4 in the wild-type FMDV strain SAT2/SAU/6/2000.

In consistency with the modification (i), also the position of modification (ii) may possibly vary slightly and may be 52 or 53, for example. To identify or confirm the amino acid which is to be modified, the amino acid sequence of this region of several FMDV strains are aligned with the corresponding region. It was found, in particular, by comparing the sequences of different FMDV strains, that the region may be written as follows:

Gly Ser Thr X₄ Thr Thr Ser;

where
X₄ is Asp
   and the modification is in X₄, which is modified in the invention to glycine.

In a preferred embodiment, the capsid precursor protein comprising the VP1 and VP4 proteins of the invention comprising the modifications (i) and (ii) is part of the full-length capsid precursor protein P1.

In a further preferred embodiment, the recombinant FMDV VP1 protein of the invention comprises the amino acid sequence of SEQ ID NO: 4, which is based on the amino acid sequence of the VP1 protein of FMDV strain SAT2/SAU/6/2000 (SEQ ID NO: 1) and including the modification (i) described above.

In a further preferred embodiment, the recombinant FMDV VP4 protein of the invention comprises the amino acid sequence of SEQ ID NO: 5, which is based on the amino acid sequence of the VP4 protein of FMDV strain SAT2/SAU/6/2000 (SEQ ID NO: 2) and including the modification (ii) described above.

In case the VP1 and VP4 proteins of the invention are part of the full-length P1 protein, the recombinant FMDV capsid precursor protein P1 preferably comprises the amino acid sequence of SEQ ID NO: 6, which is based on the amino acid sequence of the capsid precursor protein P1 of FMDV strain SAT2/SAU/6/2000 (SEQ ID NO: 3) and including the modifications (i) and (ii) described above.

The present invention also relates to the nucleic acid sequences, notably the cDNA incorporating at least the modification (i) and preferably the modifications (i) and (ii). In particular, the invention relates to the cDNA, and expression vectors incorporating them, comprising the sequence coding for the VP1 protein and optionally the VP4 protein, or the full-length capsid precursor protein P1 comprising the VP1 and the VP4 protein as described above, and which incorporate these two modifications, for example cDNA sequences coding for P1-2A, and the sequences incorporating them, for example sequences incorporating them with the sequences allowing their recombinant expression, thus being operably linked to a promoter.

The present invention also relates to the amino acid sequences encoded by these nucleic acid sequences.

In a further aspect, the invention relates to an expression vector for the recombinant expression of the nucleic acid sequence of the invention and in which the nucleic acid sequence encoding the capsid precursor protein comprising the modified VP1 protein, and optionally the modified VP4 protein, is operably linked to a promoter.

In the following, the recombinant capsid precursor protein comprising the modified VP1 protein, and optionally the modified VP4 protein, of the invention and including the modifications (i) and (ii) described above is designated as “recombinant FMDV capsid precursor protein according to the invention”. The “recombinant FMDV capsid precursor protein according to the invention” may either be expressed as single entity including all structural proteins necessary for the formation of VLPs or may be expressed as separate entities, such as by separately expressing the structural VP proteins, including the VP1 and VP4 proteins of the invention.

In vitro recombinant DNA methods known to the skilled person can be used to generate a recombinant nucleic acid molecule that encodes the capsid precursor protein according to the invention, comprising the amino acid modification (i) and optionally the amino acid modification (ii). Conveniently, this can be done by making and sub-cloning PCR fragments, or by de novo gene synthesis techniques.

A recombinant FMDV capsid precursor protein according to the invention can be obtained in a variety of ways. A variety of in vivo and in vitro expression systems are well known in the art. For example, a recombinant FMDV capsid precursor protein according to the invention can be generated by manipulation of FMDV genetic material, transfection of cDNA into appropriate host cells, or amplification of infectious FMDV virus in an appropriate host cell, e.g. BHK-21 cells.

Alternatively, a recombinant FMDV capsid precursor protein according to the invention can be produced via an in vitro cell-based expression system, as this provides advantages in respect of yields and safety. The expression system can be based on prokaryotic or eukaryotic cells: if eucaryotic, can be based on host cells from a yeast, mammalian, insect, or plant, all as described in the prior art.

A preferred in vitro expression system for the expression of a recombinant FMDV capsid precursor protein according to the invention is the Baculovirus expression system (BVES). This system uses a baculovirus expression vector, which is capable of recombinantly expressing the gene of interest in insect cells, which in the present invention is the modified FMDV capsid precursor protein.

The baculovirus expression vector can be any baculovirus expression vector capable of recombinantly expressing an FMDV capsid precursor protein under control of a promoter. The promoter is not particularly limited but may be any promoter capable of recombinantly expressing the FMDV capsid precursor protein in a baculovirus expression system. Preferred promoters for use in the baculovirus expression system of the present invention are the polyhedrin (polh) promoter (described in: Ayres M. D. et al. (1994) Virology, Vol. 2020, p. 586-605) and the p10 promoter (described in: Knebel D. et al. (1985) EMBO J. Vol. 4 (5), 1301-1306) of AcNPV. Another preferred promoter is the promoter of the orf46 viral gene of S. exigua nucleopolyhedrovirus (SeNPV) (described in M. Martinez-Solís et al. (2016) PeerJ, DOI 10.7717/peerj.2183).

The expression vector may further comprise one or more translational enhancers, which enhance the recombinant expression of the FMDV capsid precursor protein. For example, the baculovirus expression vector may comprise the two translational enhancers Syn21 and p10UTR as described in EP 20 203 373 incorporated herewith by reference in its entirety.

Baculovirus expression vectors for use in baculovirus expression systems for the recombinant expression of proteins are commercially available and are extensively used in the art for the production of proteins and virus-like particles. The systems may encompass, for example, one or more transfer plasmids used to transform cells, such as E. coli cells or insect cells, in which the baculovirus expression vector is propagated. Commercially available baculovirus expression vectors include, but are not limited to, Top-Bac® vector (ALGENEX, Spain), pFastBac® vector (Thermo Fisher Scientific, Germany), flashBAC® vector (Oxford Expression Technologies Ltd, UK) and BestBacR vector (EXPRESSION SYSTEMS, CA).

The baculovirus expression vector for use in the present invention thus may contain an expression cassette comprising the nucleic acid sequence encoding the FMDV capsid precursor protein, which is expressed in the insect cell under control of a functional promoter, and preferably including one or more translational enhancers and/or other cis-acting elements.

The nucleic acid sequence encoding the FMDV capsid precursor protein is not particular limited to a certain strain and may be of any FMDV strain belonging to serotype A, O, Asia1, SAT1, SAT2, SAT3 or C. In a particularly preferred embodiment, the FMDV capsid precursor protein according to the invention is from the SAT serotype, such as SAT1, SAT2, and SAT3 serotype, and most preferably from the SAT2 serotype.

In the present invention, the FMDV capsid precursor protein may comprise all elements necessary for the processing and assembly of VLPs. Hence, the FMDV capsid precursor protein typically comprises at least the capsid precursor P1 and preferably further comprises the 2A peptide. The 2A peptide is able to release P1-2A from any downstream protein sequence.

In a further preferred embodiment, the baculovirus expression vector further comprises a nucleic acid sequence encoding a protease capable of cleaving an FMDV capsid precursor protein. The protease may be any protease capable of cleaving the FMDV capsid precursor protein as a step in the production and assembly of FMDV VLP. As mentioned above, for FMDV, proteolytic processing of the capsid precursor P1 according to the invention into VP0 (VP2 plus VP4), VP3 and VP1 occurs by means of the picornavirus 3C protease or its precursor 3CD. Hence, the protease is preferably the 3C protease of FMDV. The sequence of FMDV wild-type 3C protease from an FMDV type A strain is described in the art and is disclosed in WO 2011/048353, which is hereby incorporated by reference in its entirety. The 3C protease may also be a functional derivative including one or more mutations, which reduce its proteolytic activity, for example a mutation at cysteine 142.

The capsid precursor protein of the invention is typically cleaved by the 3C protease into VP0, VP3 and VP1. Most preferably, the baculovirus expression system expresses a P1-2A-3C cassette, i.e. it simultaneously expresses the coding regions for the proteins P1, 2A and 3C. Expression of the 3C enzyme in a P1-2A-3C cassette allows expression and processing of the P1-2A precursor into the structural proteins which assemble into VLPs. The capsid precursor protein and the protease may be expressed under control of individual promotors or under control of the same promoter. As described above, the capsid precursor proteins required for the assembly of FMDV VLPs may be split up into multiple expression units and expressed separately, for example by recombinantly producing VP1, VP2, VP3 and VP4, or recombinantly producing VP0, VP1 and VP3. In this alternative embodiment, a proteolytic cleavage of a capsid precursor protein by a 3C protease may not be necessary.

Cleavage of the capsid precursor protein or VLP may be analysed using techniques known in the art. For example, extracts from baculovirus-infected host cells may be analyzed by gel-electrophoresis and the separated proteins transferred onto a nitrocellulose membrane for Western blotting. Western blotting with protein-specific antibodies should reveal the degree of protease-mediated cleavage. For example, for FMDV, the unprocessed capsid precursor protein (P1-2A) would appear as a band of around 81 kDa, and cleavage may produce VP3-VP1 (˜47 kDa), VP0 (˜33 kDa), VP2 (˜22 kDa), VP3 (˜24 kDa) and/or VP1 (˜24 kDa).

### Method of Producing Virus Like Particles

The method for recombinantly producing the modified capsid precursor protein of the invention includes the culturing of host cells under conditions suitable for the host cell to recombinantly express the capsid precursor protein from the expression vector in order to produce VLPs. In case of using the BVES, the host cell may be an insect cell and the expression vector is a baculovirus expression vector.

The term “the host cell is capable of recombinantly producing the FMDV VLP” thus means that the insect cell can be used as a host cell for the production of recombinant capsid precursor proteins, which assemble into VLPs.

The first step of the method of the invention comprises infecting a host cell, for example an insect cell, with the expression vector, for example a baculovirus expression vector (step (i) of the method of the invention). In the preferred embodiment, the insect cell my be any insect cell, which is capable of producing FMDV VLPs in cell culture. In particular, the insect cell may be a Sf9 cell (a clonal isolate of Spodoptera frugiperda Sf21 cells), or a Tni cell (ovarian cells isolated from Trichoplusia ni). Most preferably, the host cell is a Tni cell, or a Tni-derived cell line, such as a Tnao38 cell.

Methods of infecting an insect cell with a baculovirus expression vector for the recombinant expression of proteins are known to the skilled person and are described, for example, in L. King, The Baculovirus Expression System, A laboratory guide: Springer, 1992: Baculovirus and Insect Cell Expression Protocols, Humana Press, D. W. Murhammer (ed.) 2007: Baculovirus Expression Vectors: A Laboratory Manual, Oxford University Press, D. R. O'Reilly, 1993. In the method of the invention, culturing of the insect cell is performed in cell culture medium (step (ii) of the method of the invention). Cell culture of infected insect cells under conditions under which the insect cell produces the FMDV VLP is established in the art and can be performed, for example, as described in (Porta et al., 2013, J. Virol. Methods, vol. 187, p. 406; A. C. Mignaqui et al., 2019, Critical Reviews in Biotechnology, vol. 39 (3), p. 306-320).

After culturing, the cells may optionally be separated from the cell culture to obtain culture supernatant. The term “supernatant” thus relates to the cell culture medium from which the insect cells have been removed. Recombinant proteins that are trapped inside insect cells can be released by cell disruption techniques known in the art. The obtained cell lysate contains all the cellular components and debris, and often requires laborious purification to obtain the recombinant protein in a purer form. Further, cell disruption techniques also release a lot of unwanted cellular proteins, such as proteases, which can degrade the desired proteins, thereby reducing protein yield and quality.

Conventional techniques for separation of the cells from the cell culture medium are well known in the art and include one or more of filtration, centrifugation, and sedimentation.

In step (iii) of the method of the present invention, the FMDV VLPs produced by the host cells are harvested from the cell culture and optionally are further purified. Harvesting may include the separation of the VLPs from the cells and/or culture medium and, if necessary, further purification of the VLPs. Harvesting can be performed by one or more techniques including precipitation of the VLPs with for example polyethylene glycol (PEG), affinity chromatography, or molecular sieve chromatography.

### Vaccines and Production Thereof

As described above, the preferred utility of the embodiments of the present invention is in veterinary medical use, in particular for vaccination against FMD. The present invention thus further relates to the production of FMDV VLPs obtained from the modified capsid precursor protein of the invention, and which are used in the production of a vaccine. In a preferred embodiment, the vaccine of the invention comprises FMDV VLPs produced from the modified capsid precursor protein, which is from a FMDV strain of the SAT2 serotype.

In particular, the VLPs obtained from the modified capsid precursor protein and produced by the method according to the invention may be used as antigen for vaccination of subjects. Preferably, the VLPs are incorporated into a composition comprising the VLPs and one or more pharmaceutically acceptable carrier.

The present invention thus also provides a method for the production of a vaccine, which comprises the step of producing FMDV VLPs by a method as described above and incorporating the FMDV VLPs in a vaccine, such as by the addition of a pharmaceutically acceptable carrier.

Pharmaceutically acceptable carriers are well-known in the art. Merely as an example: such a carrier can be as simple as sterile water or a buffer solution such as PBS. The vaccine may comprise a single carrier or a combination of two or more carriers. The vaccine may also comprise one or more pharmaceutically acceptable diluents, adjuvants and/or excipients. The vaccine may also comprise, or be capable of expressing, another active agent, for example one which may stimulate early protection prior to the VLP-induced adaptive immune response. The agent may be an antiviral agent, such as type I interferon. Alternatively, or in addition, the agent may be granulocyte-macrophage colony-stimulating factor (GM-CSF).

The vaccine may be used therapeutically, to treat an existing FMDV infection (especially in herds or regions where the virus is endemic), but preferably is used prophylactically, to block or reduce the likelihood of FMDV infection and/or prevent or reduce the likelihood of spreading the disease.

Many commercially available FMD vaccines are multivalent to provide protection against the different FMD serotypes. By the same token, the vaccine of the present invention may comprise a plurality of different VLPs, each directed at a different serotype and/or different subtypes within a given serotype.

Thus, in a further preferred embodiment, the method of the invention further comprises the step (iv) of incorporating the FMDV VLPs into a vaccine by addition of a pharmaceutically acceptable carrier.

The vaccine obtained by the method as described above may be used in the protection of a subject against an infection with FMDV.

The present invention also provides a method of protecting a subject against an infection with FMDV by administration of an effective amount of a vaccine of the present invention. A method of protecting a subject against an infection with FMDV comprises the step of producing an FMDV VLP by a method as described above, incorporating the VLP into a vaccine by addition of a pharmaceutically acceptable carrier, and administering the vaccine to the subject.

For FMD the subject may be a cloven-hoofed animal. FMD susceptible animals include cattle, sheep, pigs, and goats among farm stock, as well as camelids (camels, llamas, alpacas, guanaco and vicuna). Some wild animals such as hedgehogs, coypu, and any wild cloven-footed animals such as deer and zoo animals including elephants are also susceptible to FMD.

### Administration

The present invention contemplates at least one administration to an animal of an efficient amount of the vaccine according to the invention. A vaccine can be administered in any art-known method, including any local or systemic method of administration. Administration can be performed e.g. by administering the antigens into muscle tissue (intramuscular, IM), into the dermis (intradermal, ID), underneath the skin (subcutaneous, SC), underneath the mucosa (submucosal, SM), in the veins (intravenous, IV), into the body cavity (intraperitoneal, IP), orally, anally etc. For the current vaccine IM, ID and SC administration are preferred.

### EXAMPLES

The invention will be further described by way of the following non-limiting examples, which are meant to serve to assist one of ordinary skill in the art in carrying out the invention.

### BRIEF DESCRIPTION OF FIGURES

FIG. 1: Schematic representation of FMDV genome encoding a single open reading frame (ORF) that produces a precursor polyprotein that is processed into twelve mature viral proteins.

FIG. 2: Quantification by ELISA of the SAT2/ETH/65/2009 VLP concentration in insect cell lysates in Example 1.

FIG. 3: Western blot analysis of the cell lysate containing SAT2/SAU/6/2000 VLP in Example 2.

FIG. 4: EM image of SAT2/SAU/6/2000-VP2-K093C (left) and VP1-T012N+VP4-D053G (right) VLPs derived from insect cells in Example 2.

FIG. 5: FMD clinical scores in the single dose protection study in Example 3.

FIG. 6: Results of detection of viraemia by real-time RT-PCR in Example 3. The dotted line indicates the cut-off value for sample positivity.

FIG. 7: Virus neutralizing titre prior to challenge (21 dpv: 0 dpc) in Example 3.

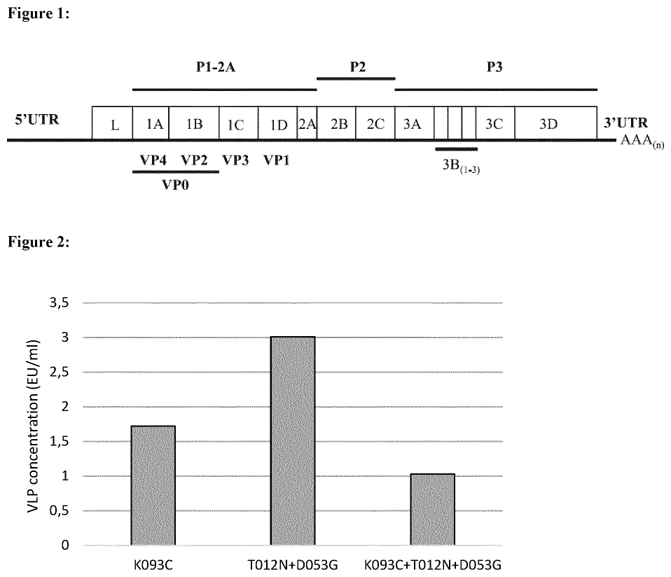

### PREPARATION OF BACULOVIRUS CONSTRUCTS

Recombinant baculoviruses were generated using the ProEasy™ system from AB Vector. They were equipped with the P1-2A-3Cpro expression cassette as described by Porta et al., 2013, J Virol Methods. To increase expression levels the so-called Syn21 translational enhancer was placed in front of the P1-2A-3Cpro open reading frame, and downstream of the P1-2A-3Cpro coding region the 3′-UTR from the Autographa californica nucleopolyhedrovirus (AcNPV) p10 gene (P10UTR) was inserted (Liu et al., 2015, Biotechnol Lett).

Since wild-type capsids cannot be expressed due to their inherent instability, the previously described modification VP2-S093C in VP2 was introduced in the P1 coding sequence as described in WO 2002/000251 (corresponding to VP2-K093C in SAT2 strains). As an alternative to this previously described mutation, two novel mutations were introduced in P1: VP1-T012N or VP4-D053G alone, or combined. In another construct the mutations were combined resulting in VP2-K093C+VP1-T012N+VP4-D053G.

The amino acid modifications were introduced using synthetic cDNA which was placed in a transfer vector used for producing the recombinant baculoviruses. The VP1-T012N mutation refers to a threonine (T) to asparagine (N) amino acid mutation at position 012 in VP1. The VP4-D053G mutation refers to an aspartate (D) to glycine (G) amino acid mutation at position 053 in VP4. The VP2-K093C mutation refers to a lysine (K) to cysteine (C) amino acid mutation at position 093 in VP2 and is as described in WO 2002/000251.

The following baculovirus expression constructs were used in the following examples for the recombinant production of VLPs in insect cells:
i) Expression construct SAT2/ETH/65/2009-VP2-K093C containing the P1-2A-3Cpro expression cassette based on FMDV strain SAT2/ETH/65/2009.
ii) Expression construct SAT2/ETH/65/2009-VP1-T012N+VP4-D053G containing the P1-2A-3Cpro expression cassette based on FMDV strain SAT2/ETH/65/2009.
iii) Expression construct SAT2/ETH/65/2009-VP2-K093C+VP1-T012N+VP4-D053G containing the P1-2A-3Cpro expression cassette based on FMDV strain SAT2/ETH/65/2009.
iv) Expression construct SAT2/SAU/6/2000-VP2-K093C containing the P1-2A-3Cpro expression cassette based on FMDV strain SAT2/SAU/6/2000.
v) Expression construct SAT2/SAU/6/2000-VP4-D053G containing the P1-2A-3Cpro expression cassette based on FMDV strain SAT2/SAU/6/2000.
vi) Expression construct SAT2/SAU/6/2000-VP1-T012N containing the P1-2A-3Cpro expression cassette based on FMDV strain SAT2/SAU/6/2000.
vii) Expression construct SAT2/SAU/6/2000-VP1-T012N+VP4-D053G containing the P1-2A-3Cpro expression cassette based on FMDV strain SAT2/SAU/6/2000.

The baculovirus expression system was used to recombinantly express the SAT2 VLPs.

### Example 1: Stabilization of SAT2/ETH/65/2009 VLPs

Erlenmeyers containing 40 ml of 1·10⁶ Tnao38 insect cells per ml were inoculated with 1 ml of a P1 baculovirus stock and incubated at 27.5° C. for 5 days post infection (dpi). The cells were collected by spinning them down for 5 min at 3000 rpm. The resulting cell pellet was resuspended in 50 mM HEPES pH 8.0-100 mM KCl (HEPES-KCl) with a volume of 1/10 of the original culture volume and cells were lysed by sonication.

The amount of intact VLPs in the material was determined by ELISA using VHH M377F (Harmsen et al., 2017, Front. Immunol. 8:960, doi: 10.3389/fimmu.2017.00960). Serially diluted samples were incubated for 1 h at room temperature (RT) on microtiter plates coated overnight at 4° C. with antibody. After removing the samples and three washes with PBS-Tween, a fixed amount of a biotinylated version of the coating antibody was added to plates and incubated for 1h at RT. The biotinylated antibody was removed and plates were washed three times with PBS-Tween, after which peroxidase-conjugated streptavidin was added to the plates followed by chromophoric detection. The VLP concentration was expressed as ELISA Units per ml (EU/ml).

In each of the three harvests VLPs could be detected by ELISA (see FIG. 2). The results suggest that a higher yield was obtained with the double mutant (VP1-T012N+VP4-D053G). It also seems that the combination of VP2-K093C and the double mutant (triple mutant: VP2-K093C+VP1-T012N+VP4-D053G) is not beneficial in terms of VLP yield.

The obtained material was heat treated at 46° C. for 20 minutes and the amount of intact VLPs was determined by ELISA before and after heat treatment. The percentage of capsids that survived the incubation at 46° C. is shown in Table 1. From this data it can be concluded that the double mutant is more heat resistant than the VP2-K093C VLP. A synergistic effect is not observed for the triple mutant that has all three stabilizing mutations, since the VP2-K093C+VP1-T012N+VP4-D053G VLP is less heat stable than the double mutant, although more stable than the VLP with VP2-K093C alone.

TABLE 1
Thermostability of mutant SAT2/ETH/65/2009 VLPs

| | Intact VLPs after |
|---|---|
| | heat treatment |
| SAT2/ETH/65/2009 VLP | (20 min; 46° C.) |
| VP2-K093C | 16% |
| VP1-T012N + VP4-D053G | 42% |
| VP2-K093C + VP1-T012N + | 24% |
| VP4-D053G | |

Overall, the data presented in this example indicates that VP1-T012N+VP4-D053G outperforms VP2-K093C in terms of yield and thermostability, both important parameters for the development of a vaccine against FMD.

### Example 2: Stabilization of SAT2/SAU/6/2000 VLPs

To further investigate if the VP1-T012N+VP4-D053G combination can stabilize VLPs of other strains belonging to the SAT2 serotype, a new set of recombinant baculoviruses with a P1-2A-3Cpro expression cassette based on strain SAT2/SAU/6/2000 was generated following the method described above.

The following stabilizing mutations were introduced: VP2-K093C, VP1-T012N+VP4-D053G, VP1-T012N and VP4-D053G, the latter two to understand what the individual contribution is of the mutations in VP1-T012N+VP4-D053G.

Four 2-liter bioreactors containing 2·10⁶ Tnao38 insect cells per ml were inoculated at MOI=1 with recombinant baculoviruses. At 4 days post infection the cell pellet obtained after centrifugation was sonicated in 100 ml of HEPES-KCl buffer (concentration factor: 20×). Cell culture supernatant was also collected at 4 dpi.

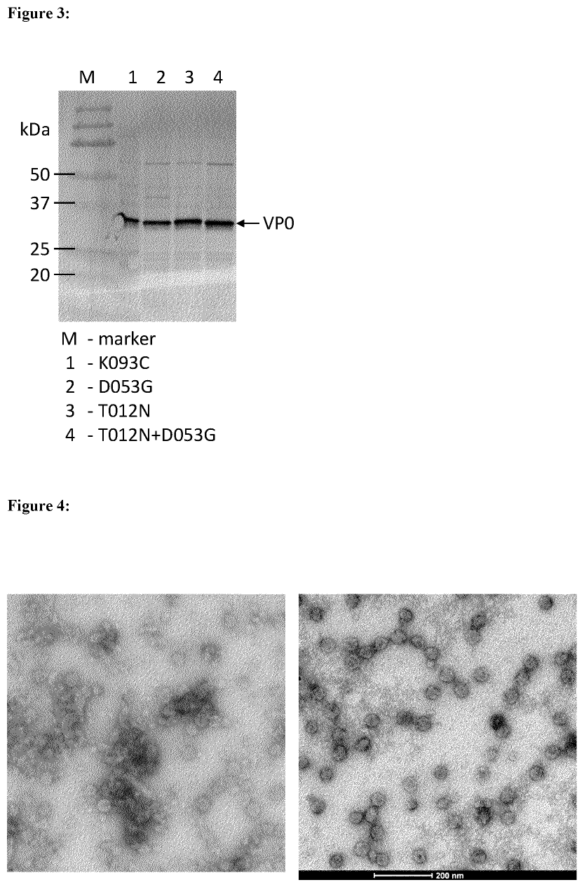

The concentrated cell pellets were pre-diluted 40 times and analyzed by Western blotting using the anti-VP2 monoclonal antibody F1412SA (Yang et al., 2007, Vet Immunol Immunopathol). This antibody detected the VP0 protein, which is a precursor of VP4 and VP2 (FIG. 3). Visual inspection of the VP0 band on the Western blot suggests that the expression level of the VP4-D053G mutant is slightly less than the other three mutants (i.e. VP2-K093C, VP1-T012N and VP1-T012N+VP4-D053G).

In each of the four harvests VLPs could be detected by ELISA using the method as described in example 1 (Table 2). The highest yield in the cell lysate was obtained with the double mutant (VP1-T012N+VP4-D053G) and the lowest yield was achieved with the VP4-D053G confirming the Western blot results. The ELISA data also suggests that the single VP1-T012N and VP4-D053G mutants form capsids that can be recognized by the intact-capsid specific M377F antibody. Surprisingly, VP2-K093C capsids could not be detected in the cell culture supernatant, while the other mutant VLPs were present in the cell culture supernatant. This observation could indicate that the capsids in the supernatant have matured and were actively transported to the extracellular environment, such as is the case for FMDV in naturally infected cells.

The VLPs in the cell culture supernatant resist the relative low pH of approximately 6.5 of the insect cell medium, which is detrimental to SAT virions (Scott et al, 2019, Virus Res 264:45-55, doi.org/10.1016/j.virusres.2019.02.012). The overall yield per millilitre cell culture is for the VP1-T012N and the double mutant significantly higher than for VP2-K093C and VP4-D053G.

The obtained material was heat treated at 37° C. for 25 minutes and the amount of intact VLPs was determined before and after heat treatment by ELISA. The percentage of capsids that survived the incubation at 37° C. is shown in Table 2.

From this data it can be concluded that the VP1-T012N mutant and VP1-T012N+VP4-D053G double mutant are more heat resistant than the VP2-K093C VLP.

To demonstrate that intact capsids were present the cell lysates were subjected to sucrose banding and fractions were analysed by electron microscopy (EM). Only for VP2-K093C and VP1-T012N+VP4-D053G, a significant number of (icosahedral) capsids of about 30 nm could be identified (FIG. 4).

In summary, the data obtained with SAT2/SAU/6/2000 VLPs shows that T012N+D053G VLPs are formed and can be expressed at a higher yield than K093C VLPs and are more thermostable.

TABLE 2
Yield of stabilized SAT2/SAU/6/2000 VLPs in
both cell culture supernatant and cell lysate

| | VLP concentration (EU/ml) | | | Percent intact |
|---|---|---|---|---|
| | Cell | | Sum | VLPs after |
| SAT2/SAU/6/ | culture | 20× | (per ml | heat treatment |
| 2000 VLP | supernatant | cell lysate | culture) | (25 min; 37° C.) |
| VP2-K093C | 0.0 | 34.1 | 1.7 | 39% |
| VP4-D053G | 0.5 | 29.1 | 1.9 | n.d. |
| VP1-T012N | 1.4 | 37.2 | 3.3 | 82% |
| VP1-T012N + | 1.0 | 40.4 | 3.1 | 82% |
| VP4-D053G | | | | |

n.d.—not determined

### Example 3: Animal Trial with SAT2/SAU/6/2000 VP1-T012N+VP4-D053G VLP

An animal trial was performed to demonstrate that the VP1-T012N+VP4-D053G VLPs are immunogenic and that vaccines containing these VLPs can protect cattle against homologous FMDV challenge. Seventeen calves, 4-6 months old, were grouped in 3 groups containing 5 calves each (vaccine groups) and in one group of 2 (control group). On day 0, calves in group 1, 2, and 3 were vaccinated intramuscularly (IM) with 2 ml of SAT2/SAU/6/2000 vaccines containing an adjuvant that forms a so-called double oil emulsion (DOE) after mixing with the water phase of the vaccine. Vaccines were formulated with Montanide ISA 206 VG (Seppic, France) following the recommendations of the supplier. Group 1 animals received a vaccine containing VP2-K093C VLPs, group 2 animals received a vaccine containing VP1-T012N+VP4-D053G VLPs, and the animals in group 3 received a vaccine containing inactivated FMDV SAT2/SAU/6/2000 (“classic vaccine”). On day 21, all calves were challenged by the intradermolingual (IDL) route with 1·10⁵ TCID50 of FMDV strain SAT2/SAU/6/2000. Blood samples were taken at 21 days post vaccination (dpv) as well as at 0 to 6 days post challenge (dpc). The virus neutralization titre (VNT) in blood was determined by VN assay and the FMDV virus load in blood was determined by real-time RT-PCR. Animals were inspected and clinically scored daily for clinical signs (i.e. FMD lesions and fever). An overview of the groups and their treatment is presented in Table 3.

TABLE 3
Animal groups and treatment of the SAT2/SAU/6/2000 vaccination-challenge study.

| | Vaccine | | | | Challenge | |
|---|---|---|---|---|---|---|
| Group | SAT2/SAU/6/2000 | Antigen | | Route; | FMDV | Route; |
| (n) | vaccine | payload (μg) | Adjuvant | dose | strain | dose |
| 1 (5) | VLP − VP2 − K093C | 10 | Montanide | IM; 2 ml | SAT2/SAU/6/2000 | IDL; 1 · 10⁵ TCID₅₀ |
| | | | ISA 206 VG | | | |
| 2 (5) | VLP − VP1 − | | | | | |
| | T012N + VP4 − D053G | | | | | |
| 3 (5) | Classic | | | | | |
| 4 (2) | — | — | — | — | | |

The VP2-K093C and VP1-T012N+VP4-D053G VLPs in the vaccines used for group 1 and 2, respectively, were derived from the 20× cell lysate concentrate prepared in example 2.

The inactivated virus in the classic vaccine used for group 3 was prepared as follows. Twenty roller bottles containing BHK-21 cells were infected with FMDV SAT2/SAU/6/2000. Virus was harvested and centrifuged at 3,000×g for 10 minutes. Supernatant was inactivated twice with binary ethylenimine (BEI) for 24 hours at 37° C. BEI inactivated FMDV was precipitated with ammonium sulphate at +4° C. overnight. After centrifugation (3,000×g for 45 minutes), FMDV was resuspended in sterile Dulbecco's phosphate buffered saline (DPBS) and a total final concentration of 1% IGEPAL was added. This was incubated on ice for 20 minutes prior ultracentrifugation at 20,000 rpm for 20 minutes at 12° C. The supernatant was decanted into fresh ultracentrifuge tubes and underlayed with 2 ml 20% sucrose. After ultracentrifugation (2.5h at 28,000 rpm, 12° C.), the pellets were resuspended in DPBS and tested for innocuity by virus isolation assay.

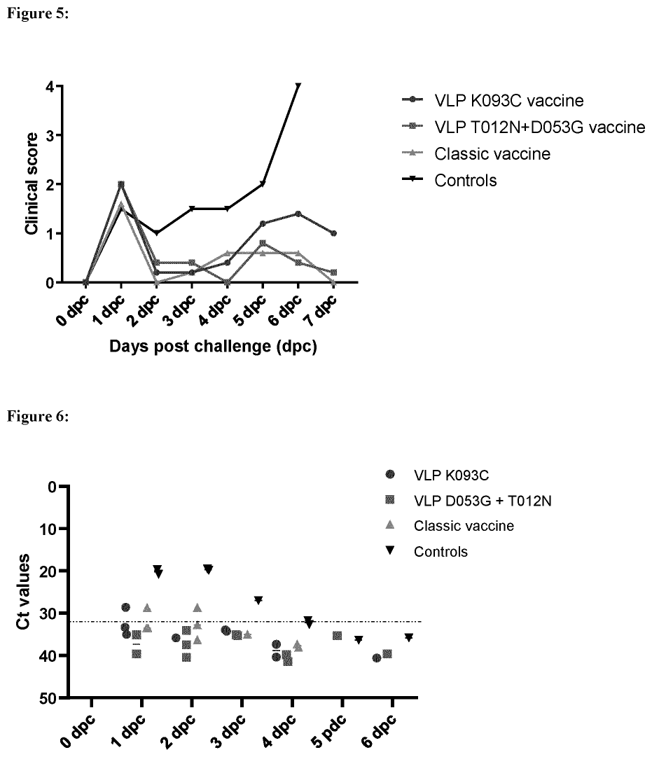

At the end of the experiment, all vaccinated calves were protected against foot lesions. At 6 dpc, one calf (out of two) from the control group showed lesions on its feet. The other calf from the control group died unexpectedly at 5 dpc possibly due to myocarditis. Both calves in the control group showed secondary FMDV replication in mouth and had developed nasal discharge at the end of the study. The average clinical signs per group during the challenge period shows that the animals in the control group had overall higher clinical scores than the vaccinated animals (FIG. 5).

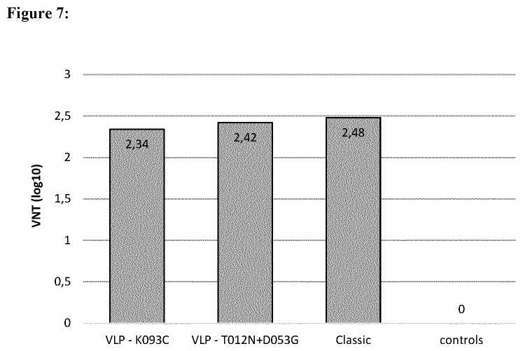

In the non-vaccinated control group, both calves developed early and high FMDV viraemia (at 1 dpc) with a threshold cycle (Ct) of 20 in RT-PCR. In the classic vaccine group, only one animal showed low viremia (Ct=28) between 1-2 dpc. In the VLP VP2-K093C single mutant group only 2 out of 5 animals showed low viremia (Ct=28). In the VLP VP1-T012N+VP4-D053G double mutant group, none of the animals were positive for RNA in blood above the cut-off value (FIG. 6).

All animals in the vaccinated groups developed high levels of VNT antibodies prior to challenge (FIG. 7). Controls animals did not seroconvert as expected. There was no significant difference between the VLP vaccines and the classic vaccine groups (p>0.05).

From the animal trial it can be concluded that the VP2-K093C and VP1-T012N+VP4-D053G stabilized VLP are immunogenic and can induce high levels of neutralizing antibodies and can protect animals against FMDV challenge.

### CONCLUSIONS

In the present invention, it could be shown that the VP1-T012N modification in the amino acid sequence of the capsid precursor protein, in particular when used in combination with the VP4-D053G modification, results in a virus-like particle mutant (VP1-T012N or VP1-T012N+VP4-D053G) that is significantly more thermostable than the prior art mutant VP2-K093C and gives higher expression levels. The VLPs derived from the double mutant capsid precursor protein are immunogenic and can be used for the vaccination of subjects providing protection against an infection with FMDV.

1. A recombinant foot and mouth disease virus (FMDV) capsid precursor protein, comprising at least the virus protein VP1, wherein the VP1 amino acid sequence is modified:
(i) by replacement of amino acid 12 of the amino acid sequence as set forth in SEQ NO: 1 or of the amino acid corresponding to amino acid 12 of the amino acid sequence as set forth in SEQ NO: 1 by an asparagine.

2. The recombinant FMDV capsid precursor protein according to claim 1, wherein the amino acid sequence of the capsid precursor protein further comprises the virus protein VP4, wherein the VP4 amino acid sequence is modified:
(ii) by replacement of amino acid 53 of the amino acid sequence as set forth in SEQ NO: 2 or of the amino acid corresponding to amino acid 53 of the amino acid sequence as set forth in SEQ NO: 2 by a glycine.

3. The recombinant FMDV capsid precursor protein according to claim 1, which comprises at least the capsid precursor protein P1.

4. The recombinant FMDV capsid precursor protein according to claim 1, which comprises the amino acid sequence of SEQ ID NO. 6.

5. An isolated nucleic acid encoding recombinant FMDV capsid precursor protein according to claim 1.

6. An expression vector comprising the nucleic acid sequence according to claim 5 operably linked to a promoter.

7. The expression vector according to claim 6, which is a baculovirus expression vector.

8. The expression vector according to claim 7, further comprising a nucleic acid sequence encoding a protease capable of cleaving the P1 capsid precursor protein into one or more capsid proteins.

9. The expression vector according to claim 7, wherein the capsid precursor protein comprises the capsid precursor protein P1 and the 2A peptide and the protease is 3C.

10. The expression vector according to claim 6, wherein the FMDV is of the SAT2 serotype.

11. A method of producing FMDV virus-like particles (VLP) in a recombinant expression system, the method comprising:
(i) infecting a host cell with an expression vector according to claim 6, wherein the host cell is capable of recombinantly producing the VLP,
(ii) culturing the host cell under conditions under which the host cell produces the FMDV VLP, and
(iii) harvesting FMDV VLP produced by the host cell from the cell culture.

12. The method according to claim 11, wherein the host cell is an insect cell.

13. The method according to claim 11, the method further comprising:
(iv) incorporating the FMDV VLP into a vaccine by addition of a pharmaceutically acceptable carrier.

14. A vaccine for use in the protection of a subject against an infection with FMDV, the vaccine being obtainable by a method according to claim 13.

15. A method of protecting a subject against an infection with FMDV, which comprises the step of producing an FMDV VLP by a method according to claim 1, incorporating the VLP into a vaccine by addition of a pharmaceutically acceptable carrier, and administering the vaccine to the subject.

16. A vaccine comprising a FMDV VLP produced from a recombinant capsid precursor protein according to claim 1.

17. The vaccine according to claim 16, wherein the recombinant capsid precursor protein is from a FMDV of the SAT2 serotype.


SEQUENCE LISTING (6 sequences)

SEQ ID NO: 1 - protein, 214 aa, Foot-and-mouth disease virus - type SAT 2
  SAT2/SAU/6/2000 GenBank AY297948.1 VP1 wild type
TTSAGESADVVTTDPSTHGGNVQEGRRKHTEVAFLLDRSTHVHTNKTSFVVDLMDTKEKALVGAILRASTYYFCDLEIACVGDHTRAFWQPNGAPRTTQLGDNPMVFAKGGVTRFAIPFTAPHRLLSTVYNGECVYKKTPTAIRGDRAALAVKYADSTHTLPSTFNFGFVTVDKPVDVYYRMKRAELYCPRPLLPAYEHTGGDRFDAPIGVERQ

SEQ ID NO: 2 - protein, 85 aa, Foot-and-mouth disease virus - type SAT 2
  SAT2/SAU/6/2000 GenBank AY297948.1 VP4 wild type
GAGQSSPATGSQDQSGNTGSIINNYYMQQYQNSMDTQLGDNAISGGSNEGSTDTTSTHTNNTQNNDWFSKLAQSAISGLFGALLA

SEQ ID NO: 3 - protein, 713 aa, Foot-and-mouth disease virus - type SAT 2
  SAT2/SAU/6/2000 GenBank AY297948.1 P1 wild type
MGAGQSSPATGSQDQSGNTGSIINNYYMQQYQNSMDTQLGDNAISGGSNEGSTDTTSTHTNNTQNNDWFSKLAQSAISGLFGALLADKKTEETTLLEDRILTTRHGTTTSTTQSSVGVTLGYADADSFRPGPNTSGLETRVQQAERFFKEKLFDWTSDKPFGTLYVLELPKDHKGIYGKLTDSYTYMRNGWDVQVSATSTQFNGGSLLVAMVPELSSLKSREEFQLTLYPHQFINPRTNTTAHIQVPYLGVNRHDQGKRHHAWSLVVMVLTPLTTEAQMNSGTVEVYANIAPTNVVVAGELPGKQGIVPVAAADGYGGFQNTDPKTADPIYGYVYNPSRNDCHGRFSNLLDVAEACPTLLDFDGKPYIVTKNNGDKVMTSFDVAFTHKVHRNTFLAGLADYYTQYSGSLNYHFMYTGPTHHKAKFMVAYVPPGVETAQLPTTPEDAAHCYHAEWDTGLNSSFSFAVPYISAADFSYTHTDTPAMATTNGWVIVLQVTDTHSAEAAVVVSVSAGPDLEFRFPIDPVRQTTSAGESADVVTTDPSTHGGNVQEGRRKHTEVAFLLDRSTHVHTNKTSFVVDLMDTKEKALVGAILRASTYYFCDLEIACVGDHTRAFWQPNGAPRTTQLGDNPMVFAKGGVTRFAIPFTAPHRLLSTVYNGECVYKKTPTAIRGDRAALAVKYADSTHTLPSTFNFGFVTVDKPVDVYYRMKRAE

SEQ ID NO: 4 - protein, 214 aa, Foot-and-mouth disease virus - type SAT 2
  SAT2/SAU/6/2000 GenBank AY297948.1 VP1 mutant
TTSAGESADVVNTDPSTHGGNVQEGRRKHTEVAFLLDRSTHVHTNKTSFVVDLMDTKEKALVGAILRASTYYFCDLEIACVGDHTRAFWQPNGAPRTTQLGDNPMVFAKGGVTRFAIPFTAPHRLLSTVYNGECVYKKTPTAIRGDRAALAVKYADSTHTLPSTFNFGFVTVDKPVDVYYRMKRAELYCPRPLLPAYEHTGGDRFDAPIGVERQ

SEQ ID NO: 5 - protein, 85 aa, Foot-and-mouth disease virus - type SAT 2
  SAT2/SAU/6/2000 GenBank AY297948.1 VP4 modified
GAGQSSPATGSQDQSGNTGSIINNYYMQQYQNSMDTQLGDNAISGGSNEGSTGTTSTHTNNTQNNDWFSKLAQSAISGLFGALLA

SEQ ID NO: 6 - protein, 741 aa, Foot-and-mouth disease virus - type SAT 2
  SAT2/SAU/6/2000 GenBank AY297948.1 P1 double mutant
MGAGQSSPATGSQDQSGNTGSIINNYYMQQYQNSMDTQLGDNAISGGSNEGSTGTTSTHTNNTQNNDWFSKLAQSAISGLFGALLADKKTEETTLLEDRILTTRHGTTTSTTQSSVGVTLGYADADSFRPGPNTSGLETRVQQAERFFKEKLFDWTSDKPFGTLYVLELPKDHKGIYGKLTDSYTYMRNGWDVQVSATSTQFNGGSLLVAMVPELSSLKSREEFQLTLYPHQFINPRTNTTAHIQVPYLGVNRHDQGKRHHAWSLVVMVLTPLTTEAQMNSGTVEVYANIAPTNVVVAGELPGKQGIVPVAAADGYGGFQNTDPKTADPIYGYVYNPSRNDCHGRFSNLLDVAEACPTLLDFDGKPYIVTKNNGDKVMTSFDVAFTHKVHRNTFLAGLADYYTQYSGSLNYHFMYTGPTHHKAKFMVAYVPPGVETAQLPTTPEDAAHCYHAEWDTGLNSSFSFAVPYISAADFSYTHTDTPAMATTNGWVIVLQVTDTHSAEAAVVVSVSAGPDLEFRFPIDPVRQTTSAGESADVVNTDPSTHGGNVQEGRRKHTEVAFLLDRSTHVHTNKTSFVVDLMDTKEKALVGAILRASTYYFCDLEIACVGDHTRAFWQPNGAPRTTQLGDNPMVFAKGGVTRFAIPFTAPHRLLSTVYNGECVYKKTPTAIRGDRAALAVKYADSTHTLPSTFNFGFVTVDKPVDVYYRMKRAELYCPRPLLPAYEHTGGDRFDAPIGVERQ